(19) United States
(12) Patent Application Publication
(10) Pub. No.: US 2007/0025918 A1
(43) Pub. Date: Feb. 1, 2007

(54) Magnetic resonance imaging (MRI) agents: water soluble carbon-13 enriched fullerene and carbon nanotubes for use with dynamic nuclear polarization

(75) Inventor: Ralph E. Hurd, Milpitas, CA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: General Electric Company, Schenectady, NY (US)

(21) Appl. No.: 11/191,034

(22) Filed: Jul. 28, 2005

(51) Int. Cl. A61K 49/10

(52) U.S. Cl. 424/9.34; 424/9.36; 977/736; 977/744

(57) ABSTRACT

The invention relates to carbon-13 enriched fullerene and carbon nanotube (CNT) compositions for improved magnetic resonance imaging (“MRI”). The invention also relates to a dynamic nuclear polarization (DNP) method of MRI utilizing the carbon-13 enriched fullerene and CNTs of the invention.

### FIELD OF THE INVENTION

The present invention relates generally to the field of magnetic resonance imaging (MRI). In particular, the present invention relates to a method of dynamic nuclear polarization with ¹³C-enriched fullerene and carbon nanotubes as (MRI) contrast agents.

### BACKGROUND OF THE INVENTION

Contrast agents have played an important role in medical imaging procedures to enhance the image contrast in images of a subject, using for example X-ray, magnetic resonance and ultrasound imaging. The resulting enhanced contrast enables different organs, tissue types or body compartments to be more clearly observed or identified. In X-ray imaging the contrast agents function by modifying the X-ray absorption characteristics of the body sites in which they distribute. Commonly used magnetic resonance contrast agents generally function by modifying the density or the characteristic relaxation times, generally of water protons, from the resonance signals of which the images are generated. And, ultrasound contrast agents function by modifying the speed of sound or the density in the body sites into which they distribute.

While the phenomenon of nuclear magnetic resonance (hereinafter NMR) was discovered in 1945, it is only relatively recently that it has found application as a means of mapping the internal structure of the body as a result of the original suggestion of Lauterbur (Nature, 242, 190-191 (1973)). In 2003, Lauterbur and Mansfield received the Nobel Prize in physiology or medicine for their contributions to developing magnetic resonance imaging (hereinafter MRI) as a technique for 3-D imaging. MRI is a very powerful imaging tool that produces results analogous to X-ray images, but is advantageously non-invasive as it avoids the use of exposing the patient under study to harmful radiation. The fundamental lack of any known hazard associated with the level of the magnetic and radio-frequency (hereinafter RF) fields that are employed renders it possible to make repeated scans on vulnerable individuals. MRI encompasses the detection of certain atomic nuclei (those possessing magnetic dipole moments) utilizing magnetic fields and radio-frequency radiation. It is similar in some respects to X-ray computed tomography (“CT”) in providing a cross-sectional display of the body organ anatomy with excellent resolution of soft tissue detail.

MRI works by exciting the molecules of a target object using a harmless pulse of RF energy to excite NMR active nuclei that have first been aligned using a strong external magnetic field and then measuring the nuclei's rate of return to an equilibrium state within the magnetic field following termination of the RF pulse. These nuclei, as they relax, subsequently emit RF energy at a sharp resonance frequency which depends on the applied magnetic field. The decay of the emitted radiation is characterized by two relaxation times, T₁ and T₂. T₁ is the spin-lattice relaxation time or longitudinal relaxation time, i.e., the time taken by the nuclei to return to equilibrium along the direction of the externally applied magnetic field. T₂ is the spin-spin relaxation time associated with the dephasing of the initially coherent precession of individual proton spins. These relaxation times have been established for various fluids, organs, and tissues in different species of mammals. For protons and other suitable nuclei, these relaxation times are influenced by the environment of the nuclei (e.g., viscosity, temperature, and the like). These two relaxation phenomena are essentially mechanisms whereby the initially imparted RF energy is dissipated to the surrounding environment. Hence, the signal that is generated contains information on nuclear spin density, T₁ and T₂. The visually readable magnetic resonance images that are generated as output are the result of complex computer data reconstruction on the basis of these information.

Because successful imaging depends on the ability of the computer to recognize and differentiate between different types of tissue, it is routine to apply a contrast agent to the tissue prior to making the image. The contrast agent alters the response of the aligned protons or other NMR active nuclei to the RF signal. Good contrast agents interact differently with different types of tissue, with the result that the effect of the contrast agent is greater on certain body parts, thus making them easier to differentiate and image. The most common contrast agents involves the hydrogen atom, which has a nucleus consisting of a single unpaired proton, and therefore has the strongest magnetic dipole moment of any nucleus. Since hydrogen occurs in both water and lipids, it is abundant in the human body. Therefore, MRI is most commonly used to produce images based upon the distribution density of protons and/or the relaxation times of protons in organs and tissues.

Other atomic nuclei having a net magnetic dipole moment also exhibit a nuclear magnetic resonance (NMR) phenomenon which may be used in MRI applications. Such nuclei include carbon-13 (six protons and seven neutrons), fluorine-19 (9 protons and 10 neutrons), sodium-23 (11 protons and 12 neutrons), and phosphorus-31 (15 protons and 16 neutrons) etc.

Additionally, paramagnetic transition metal ions, metal complexes and chelates are NMR active and can be used in MRI. The use of paramagnetic metal ions, such as Mn (II), as contrast agents in MRI was first proposed by Lauterbur et al. in 1978. Since that time, a wide range of paramagnetic metal ion chelate complexes have been proposed. Metal ions that are reasonably stable and possess the highest magnetic moment, such as Mn²⁺, Fe³⁺, and Gd³⁺, are the most commonly employed, but any paramagnetic transition metal ion may also be suitable. More recently, the use of superparamagnetic particles as MRI contrast agents has been described in U.S. Pat. No. 4,863,715.

While metal ion contrast agents are often used in MRI, they are not suitable for all imaging applications. For example, they are not particularly useful in certain body areas such as the gastrointestinal (GI) tract. In addition, these contrast agents can be toxic and chemically reactive in vivo. Hence, the majority of contrast agent research has focused on developing non-toxic, stable chelates for binding these metal ions. Attempts have been made to achieve tissue-specific MRI contrast enhancement, to decrease toxicity, or to enhance stability and/or relaxivity by coupling of the paramagnetic chelates, or metal complexing groups, to various macromolecules or biomolecules such as polysaccharides, proteins, antibodies or liposomes. However, these metal chelates have not adequately solved the needs for non-toxic contrast agents for effective in vivo imaging.

In the search for a highly effective, non-toxic contrast agent, fullerene (“buckyballs”) molecules have received much attention. Researchers have speculated that fullerenes might be used to safely encapsulate and carry medically useful metals to different parts of the body where they could then be used for diagnostic or therapeutic purposes. For a review, see e.g. Wilson L. J., Electrochem. Soc. Interface. Winter (1999). For example, investigational new drugs (INDs) have been filed for drug candidates based on C₆₀ compounds by C sixty Inc., Houston Tex. Further, tri-metal endohedral based fullerenes, e.g., A₃N@C₈₀ for MRI studies proposed by Luna Nanomaterials, Blacksburg, Va. Moreover, Mikawa et al. Bioconjugate Chem 12, 510-514 (2001) have synthesized water soluble gadolinium (Gd) endohedral metallofullerenes as poly hydroxyl forms [Gd@C₈₂ (OH)ₙ], and their paramagnetic properties were evaluated for MRI contrast agents. However, there is a concern regarding the safety of these endohedral metallofullerenesfor in vivo studies.

Besides these metallofullerene based negative contrast agents that depend on the relaxation of water, other NMR active isotopes such as ¹⁹F fluorinated fullerene based positive contrast imaging agents have been proposed. For example, Neumann et al. in U.S. Pat. No. 5,248,498, the disclosure of which is incorporated herein by reference, disclose a perfluorinated metallofullerene, C₆₀F₆₀, for MRI studies. These contrast agents afford the possibility of conducting direct ¹⁹F MRI imaging studies. However, owing to the increased tissue toxicity due to high fluorine and metal concentrations, only a low concentration of the contrast agent can be used. As a result, the sensitivity and signal strength would be so weak as to be challenging for in vivo studies. Additionally, though C₆₀F₆₀ is soluble in organic solvents THF and acetone, it is virtually insoluble in water rendering this agent impractical as a MRI contrast agent for in vivo use.

Further, Watson et al. in U.S. Pat. No. 5,688,486 disclose using fullerene molecules as cages or carriers for diagnostic or therapeutic entities. In particular, molecules are disclosed that enclose or support metal atoms or ions, preferably those that are paramagnetic or a radioisotope or have a large x-ray cross-section. In this capacity, the fullerene would act as a carrier for a metal atom or ion and maintain the same functionality as the metal chelates. Most of the compounds disclosed in the '486 patent, and the commercially available metallofullerenes described supra, however, include undesirable and toxic metals that pose biological hazards and safety concerns.

While the '486 patent discloses that the molecular mesh compounds can be used as contrast enhancing agents in imaging modalities such as MRI, Overhauser MRI, X-ray CT, SPECT etc., the compounds disclosed in the cited reference of Krusic et al., Science, 254:1183-1185 (1991) (describing benzyl- and methyl-fullerene radicals) have very poor solubility in water. Indeed, because the compounds disclosed by Krusic et al. are insoluble in water and are prepared only under anaerobic conditions, they are ineffective as in vivo contrast agents. To be effective as in vivo contrast agents, compounds must have good solubility in water of at least 3 mM for a conventional MRI measurement without ex-vivo enhancement discussed infra.

In order to address the water solubility of the fullerene molecule as a MRI contrast agent, Alford et al. in U.S. Pat. No. 6,355,225, the disclosure of which is incorporated herein by reference, teach a fullerene radical (fullerol) contrast agent for enhancing contrast in vivo magnetic resonance measurements, comprised of a water soluble, air stable paramagnetic fullerene molecule with an unpaired electron (radical). Their approach avoids using toxic paramagnetic metal species as these compounds derive their magnetic relaxation efficacy from unpaired electrons associated with the fullerene cage. These fullerene compounds were hydroxylated to form water-soluble paramagnetic compounds that can be used as in vivo MRI contrast agents.

Although these fullerol molecules (radicals) do not require the presence of a toxic paramagnetic metal species, one of the drawbacks of the fullerol as an effective contrast reagent is that the fullerol derives its primary measurement indirectly from its water-proton relaxivity, which is a negative signal. Further, the proton relaxivity of these fullerol compounds are considerably lower (0.5 mM⁻¹sec⁻¹) than Gd-chelates (about 3.5 mM⁻¹sec⁻¹). Moreover, water proton T₁ relaxation times are inherently shorter than those of other nuclei such as carbon-13 and nitrogen-15, and therefore fullerols that use water proton measurements have an inherent limitation in performing extended imaging studies as blood pool agents. Additionally, because high concentration of the fullerol is required for imaging studies, this could also raise bio-hazard and safety concerns.

In view of the foregoing discussion, it would be desirable to provide carbon-13 enriched fullerenes and carbon nanotubes (CNTs) as MRI agents for enhancing images of body organs and tissues, which overcomes the above-described inadequacies and shortcomings in the art. Because the concentration of ¹³C in the body and tissues is not sufficiently high to produce a detectable MR signal, an external carbon-13 probe must be provided. One significant advantage in using carbon-13 enriched fullerenes and CNTs is that it involves direct measurement of the carbon-13 nucleus relaxation (positive signal) rather than the water proton relaxation (negative signal). As a result, this approach provides a non-toxic, positive contrast agent with greater sensitivity and longer time window owing to the long ¹³C has a long T₁ relaxation time for MRI studies. However, due to the ˜10³ weaker MR signal from carbon-13 compared to the proton signal, it would also be desirable to use carbon-13 enriched fullerenes and CNTs with a newly developed technique of dynamic nuclear polarization (hereinafter DNP) that will amplify the signal several orders of magnitude higher for enhanced MR imaging studies.

It has now been found that ex vivo methods of magnetic resonance imaging may be improved by using polarized MR imaging agents comprising nuclei capable of emitting magnetic resonance signals in a uniform magnetic field. For example, U.S. Pat. Nos. 6,466,814 and 6,453,188 to Ardenkjaer-Larson et al., the disclosures of which are incorporated herein by reference in their entirety, teach a method of ex-vivo DNP of a high T₁ agent such as ¹³C and ¹⁵N nuclei. Contrast enhancement was achieved by utilizing the “Overhauser effect” (also known as dynamic nuclear polarization) in which an electron spin resonance (ESR) transition in an administered paramagnetic species (hereinafter an OMRI contrast agent or DNP free radical source) is coupled to the nuclear spin system of the imaging nuclei. The Overhauser effect was shown to significantly increase the population difference between excited and ground nuclear spin states of selected nuclei and thereby amplify the MR signal intensity by a factor of a hundred or more allowing MRI images to be generated rapidly and with relatively low primary magnetic fields. The ex vivo method has inter alia the advantage that it is possible to avoid administering the whole of, or substantially the whole of, the polarizing agent to the sample under investigation, while still achieving the desired polarization. Thus, the method is less constrained by physiological factors such as the constraints imposed by the administrability, biodegradability and toxicity of DNP free radical source in other in vivo techniques.

While the '814 patent discloses small molecule for metabolic markers such as acetate, aryl compounds, sugars, pyruvates, urea, amino acids etc. for in vivo imaging using DNP, these compounds are not suitable for targeting or appropriate as blood pool agents. These small contrast agents are absorbed out of the blood fairly quickly, so that they are only effective as imaging agents for about one minute. Further, because carbon-13 enriched compounds must be administered to a subject to obtain the MR signal, high carbon-13 concentration could become toxic in MRI studies as metabolic markers. Additionally, because of the multiple resonances of the various carbon-13 labeled signals in these compounds, the analysis of those signals for imaging would be rather cumbersome. Thus, these class of carbon-13 enriched small molecules solve only a limited set of problems for in vivo imaging with DNP enhancement.

From the foregoing discussion, there still remains a need for carbon-13 enriched contrast agents with DNP enhancement for gaining higher sensitivity and effectiveness for medical use, especially in targeting and blood pool imaging, e.g., MRI cardiography or angiography. Angiography, or imaging of the blood vessels, is a common MRI procedure where these class of compounds hold tremendous potential. Therefore, it would be a significant advancement in the art to provide carbon-13 enriched fullerene and CNTs as MRI agents for enhancing images of body organs and tissues, which are non-toxic and may be administered in physiologically tolerable concentrations, and yet provide clear and enhanced signal owing to their high symmetry and long T₁ relaxation time for effective and extended diagnostic use in vivo MRI studies.

### OBJECTS OF THE INVENTION

It is an object of the present invention to provide water soluble carbon-13 enriched fullerenes and carbon nanotubes compositions for improved magnetic resonance imaging (MRI).

Further, it is an object of the present invention to use ex-vivo dynamic nuclear polarization (DNP) of the carbon-13 enriched fullerene and carbon nanotube compositions in generating about 10³ stronger carbon-13 NMR signal.

It is an additional object of the invention to provide a method for enhanced imaging and treating an area of a body or organs which employs at least one of the carbon-13 enriched fullerenes and CNTs of the invention.

### SUMMARY OF THE INVENTION

The aforementioned objects are accomplished by the present invention by providing the medical utility of water soluble carbon-13 enriched fullerene and CNT compositions for improved and exceptional in vivo magnetic resonance imaging (MRI) and spectroscopy. The MRI agents are preferably derived from the class of even-numbered carbon clusters referred to in the art as fullerenes. Fullerenes range in size from C₃₀ to C₁₀₀, with even larger clusters theoretically predicted. Similarly, CNTs with 1000 carbons are experimentally generated and other larger CNTs have been predicted. These stable closed carbon shells are extracted from the soot of vaporized carbon-13 doped or sintered graphite rods. The highly stable carbon-13 enriched fullerene compounds are marked by an icosahedral-cage structure, typified by a soccer ball. Some of the more common fullerene and CNT structures are illustrated in FIGS. 1 and 2.

One of the novel utility features of these contrast agents is realized with DNP method for enhancing contrast in in vivo medical imaging. This DNP method comprises:
(i) producing a hyperpolarized solution of carbon-13 enriched fullerene or carbon nanotubes, wherein the hyperpolarization of the sample is effected by means of a polarizing agent;
(ii) optionally separating the whole, substantially the whole, or a portion of said polarizing agent from the carbon-13 enriched fullerene or CNT;
(iii) exposing said sample to radiation of a frequency selected to excite nuclear spin transitions in an MR imaging carbon-13 nuclei of the enriched fullerene or carbon nanotubes;
(iv) detecting magnetic resonance signals from said sample; and
(v) optionally generating an image, dynamic flow data, diffusion data, and perfusion data from the detected signals.

The method further comprises enteral or parenteral administration to a warm-blooded animal a diagnostically effective composition of the hyperpolarized carbon-13 enriched fullerenes or CNTs of the invention dissolved in water or a physiologically suitable solvent. The method also comprises exposing the warm-blooded animal to a MR procedure with a hyperpolarized solution from the above DNP enhancement procedure of the diagnostically effective amount of carbon-13 enriched fullerenes or CNTs.

Additional objects and advantages of the invention will be set forth in the description which follows, and in part will be obvious from the description, or may be ascertained by practice of the invention.

### BRIEF DESCRIPTION OF THE DRAWINGS

In order to facilitate a fuller understanding of the present invention, reference is now made to the accompanying drawings, in which like elements are referenced with like numerals. These drawings should not be construed as limiting the present invention, but are intended to be exemplary only.

FIG. 1 shows structures for a carbon-13 enriched fullerene-based contrast agent in accordance with an embodiment of the present invention.

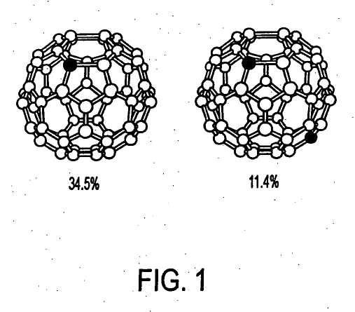

FIG. 2 shows methods of functionalization of the carbon-13 enriched fullerene and CNTs for water solubility in accordance with and embodiment of the present invention.

FIG. 3 shows an idealized ¹³C NMR spectra and structural drawings of C₆₀ (top) and C₇₀ (bottom). In C₆₀, all carbon atoms are identical and a single ¹³C NMR peak is observed. In C₇₀, there are five sets of inequivalent carbon atoms (labeled a-e), giving rise to five ¹³C NMR signals.

### DETAILED DESCRIPTION OF THE INVENTION

The particulars shown herein are by way of example and for the purposes of illustrative discussion of the embodiments of the present invention only and are presented in the cause of providing what is believed to be the most useful and readily understood description of the principles and conceptual aspects of the present invention. In this regard, no attempt is made to show structural and experimental details of the present invention in more detail than is necessary for the fundamental understanding of the present invention, the description taken with the drawings making apparent to those skilled in the art how several forms of the present invention may be embodied in practice.

The carbon allotropes useful according to the invention are fullerenes and CNTs. Discovered in 1985 fullerenes are clusters of carbon with an even number of atoms forming cage-like or tubular structures. Because of the pattern formed by the linked carbon atoms, closed cage fullerenes have been given the informal name “buckyballs” while the tubular structures have by analogy been called “buckytubes,” or alternatively, “nanotubes.” The structures were named after Buckminster Fuller, the designer of the geodesic dome.

Fullerenes are notable for their hollow polyhedral shape and their stability. The most intensively studied such carbon molecule in this class is the C₆₀ carbon cluster buckminsterfullerene in which all 60 atoms are equivalent and lie at the apices of a truncated icosahedron—the perfect soccer ball shape. C₆₀ and its discovery are described extensively in the literature—see for example Kroto et al., Nature 318: 162 (1985); Kroto, Science 242: 1139 (1988); Curl and Smalley, Science 242: 1017-1022 (1988); Kroto, Pure and Applied Chem. 62: 407-415 (1990). Many other fullerenes having stable closed cage structures have been described, e.g., C₂₈, C₃₂, C₅₀, C₇₀ (the most predominant after C₆₀), C₈₂, and the so-called “giant” fullerenes C₂₄₀, C₅₄₀, and C₉₆₀ (see, e.g., Kroto (1990), supra). The production of nested CNTs has been described for example by lijima et al. in Nature 354:56 (1991) and 356:776 (1992) and Ebbesen et al. in Nature 358:220 (1992).

The present invention involves using enriched nuclear magnetic resonance (NMR) active (I=½) carbon-13 labeled fullerenes, such as C₆₀, C₇₀, and CNTs for use as a better and new class of magnetic resonance (MRI) contrast agents. By MRI agent it is meant an agent containing nuclei (MR imaging nuclei) capable of emitting magnetic resonance signals. Generally, such nuclei will be protons, preferably water protons; however other non-zero nuclear spin nuclei may be useful (e.g. ¹⁹F, ³Li, 1 H, ¹³C, ¹⁵N or ³¹P, but preferably ¹³C nuclei) and in this event the MR signals from which the image is generated will be substantially only from the MR imaging agent (positive signal). Isotopically enriched carbon-13 fullerene contrast agents will preferably have a stronger NMR signal compared to naturally occurring fullerenes because, without the enrichment, the NMR signal is weak since the natural abundance of carbon-13 is only 1.1% and carbon-13 has a smaller gyromagnetic ratio, γ, than that of a proton (˜¼), leading to an inherently weaker NMR signal than the proton signal.

Procedures for producing fullerenes in macroscopic (multigram) quantities using electric-arc graphite decomposition are now well known and published in the literature (see Kratschmer et al., Nature 347-354 (1990); Kosh et al., J. Org. Chem. 56: 4543-4545 (1991); Scrivens et al. JACS 114:7917-7919 (1992); and Bhyrappa et al. JCS Chem. Comm. 936-937 (1992)), which are all herein incorporated by reference. Carbon-13 enriched fullerenes (or fullerene mixtures consisting primarily of C₆₀ and C₇₀) are also commercially available (e.g., MER Corp., Tucson, Ariz.; Texas Fullerene Corp., Aldrich, Tex.; Strem Chemicals, Newburyport, Mass., etc.).

The fullerene contrast agents and DNP free radical sources of this invention are preferably prepared by enriching the carbon-13 abundance of the starting material by using any method well known to those in the art, including the electric-arc graphite decomposition method, to produce the fullerenes. A known method described by Holleman et al, Chem. Phys. Lett. 240:165-171 (1995) involves doping or sintering of 13C enriched graphite rods in a DC-arc-discharge procedure to create isotopically enriched carbon-13 fullerenes. Also, carbon-13 enriched nanotubes may be produced by the method of Holleman et al.

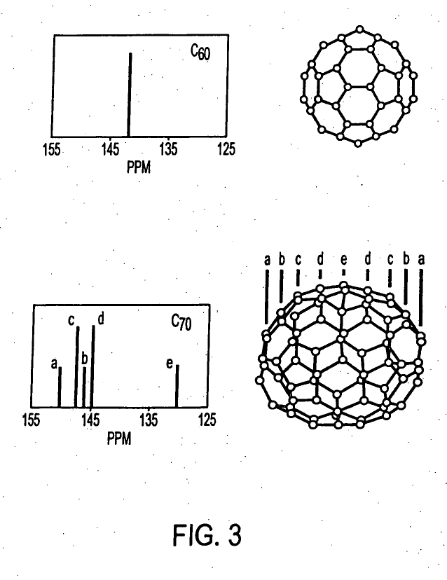

Concerning, NMR Spectra, the four-line infrared spectrum for C₆₀, as reported by Krätschmer et al. (Krätschmer & Lamb, 1990), supported the proposed truncated icosahedron structure. In addition, the ¹³C NMR spectrum of the purified C₆₀, reported by Kroto et al. (Taylor, 1990). The NMR spectrum contained a single peak at δ142.7, as expected for the highly symmetrical truncated icosahedron structure in which all carbons are identical (see FIG. 3). This result eliminated planar graphite fragments and fullerenes of lower symmetry as possible structures for C₆₀. A sixty-membered polyalkyne ring would also be expected to exhibit one ¹³C NMR signal but the observed chemical shift position (δ142.7) was inconsistent with this possibility. (Alkyne carbons generally resonate between δ50 and δ100.)

The ¹³C NMR spectrum of purified C₇₀ was also reported by Kroto and contained five peaks (FIG. 3). The proposed football-shaped C₇₀ fullerene possesses five sets of inequivalent carbon atoms in a ratio of 10:10:20:20:10. This is precisely the ratio of the line intensities observed in the ¹³C NMR spectrum.

The present invention relates to the novel utility of carbon-13 enriched fullerenes and CNTs in in vivo MRI with DNP enhancement. There are major advantages in using carbon-13 enriched fullerenes and CNT for in vivo MRI studies. Some of these advantages are analogous to the fullerene based X-ray contrast agents disclosed by Wilson et al. in U.S. Pat. No. 6,660,248, incorporated herein by reference. However, another significant advantage of the present invention is that because carbon-13 enriched fullerenes and CNTs are inherently magnetic, they do not require the presence of internal paramagnetic ions or external linkage to paramagnetic metal ions chelates or other type of magnetic functional groups to achieve their relaxation ability. Besides, these compounds do not require the measurement of water proton relaxation measurements (negative signal) because the carbon-13 relaxation is directly measured (positive signal), allowing greater sensitivity and flexibility in MRI studies.

Moreover, the high symmetry of these compounds provides additional sensitivity by generation of a single frequency response from most of the ¹³C atoms in the fullerene structure, which all have the same chemical shift for the C₆₀, C₇₀ molecules etc. Similarly, CNTs also display a single resonance frequency for CNTs, e.g., for 1000 or higher carbon counts. Other advantages include the biological compatibility, low toxicity, signal amplification through increased carbon-13 count, and long ¹³C T₁ relaxation time for extended in vivo imaging studies.

More particularly, however, recent biological studies have shown that water solubilized fullerene molecules possess unique biodistributions, and therefore they may particularly represent novel utility and advantage as blood pool imaging agents for measuring blood flow and perfusion. By changing or adding functional group(s) on the fullerene cage, it should be possible to customize the biodistribution and preferentially carry the carbon-13 enriched fullerene shell to any desired tissue in the body. Additionally, fullerene-based agents can be targeted to specific tissues by appending tissue-targeting entities (i.e., small peptides or even antibodies) to the remaining unfunctionalized surface of the C₆₀ core. See, Wilson et al. supra.

The pseudo-spherical shape is of special importance because agents with a reduced viscosity are produced, which increases the ease of injection into the body. Additionally, because C₆₀-based agents are larger than conventional contrast agents, such as iohexol, the diffusion rate through various tissues is slower. As mentioned above, this qualifies fullerene-based contrast agents as a blood pool contrast agent. Further, another advantage for blood pool imaging and angiography studies is that fullerenes and CNTs can be adjusted by the size needed, unlike small molecule based contrast agents. Therefore, these class of carbon-13 enriched fullerenes and CNTs are substantially different from previously studied fullerene-derived MRI contrast agents and represent a unique class of MRI relaxation compounds.

The commercially available carbon-13 enriched fullerenes and CNTs from the published methods, however, are not water soluble because of the hydrophobic carbon shell. Since fullerenes and CNTs exhibit extended aromaticity, chemical modification of the fullerene structure is necessary to prepare compositions suitable for in vivo applications. In order to operate effectively within a living body, the paramagnetic fullerene shell is preferably rendered water-soluble by an appropriate derivation process. This can be performed by derivatizing the fullerene shell with functional groups to impart water solubility and/or attaching the fullerene shell to a larger water-soluble molecule. The choice of functionalization method may be extremely important for obtaining the desired bio-distribution, elimination pathways, or to reduce the toxicity of the compound. Some examples and potential uses of fullerenes in biology are given by Jenson et al. (1994). Several reactions for making fullerenes water soluble are described by Hirsch (1994) in his recent review of fullerene chemistry review. Suitable methods for solubilization include but are not limited to:

Attachment of multiple hydroxyl groups using the reaction of Li et al. (1993) to produce a water soluble C₆₀(OH)₂ by reaction of C₆₀ with KOH in the presence of toluene. Fullerenes can also be polyhydroxylated using the method described by Chiang et al. (1993) and Kato et al. (2000). Polyhydroxylated fullerenes can be further derivatized using the hydroxyl (—OH) groups to form new functional groups such as esters, for example.

Further, attachment of multiple carboxylic acid groups is conveniently performed using the Bingle-Hirsch reaction to add malonic acid groups to a fullerene (reviewed by Hirsch 1994). Other methods of adding carboxylic acid groups have been reported (Isaacs and Diederich 1993). The carboxylic acid provides a convenient method (through an amide linkage) to attach the C₆₀ to other water-solubilizing functional groups.

The fullerene cage can be attached to a polypeptide (Toniolo et al. 1994), oligonucleotide, monoclonal antibody or other types of amino acid sequences.

Addition of multiple amines (reviewed by Hirsch 1994) or amino acids (Zhou et al. 1995) can also be used to solubilize the fullerene shell.

The addition of multiple alkyl sulfonates has been used to produce a water-soluble fullerene as described by Chen et al. (1998).

The fullerene can be attached to water-soluble polymers such as PEG (polyethylene glycol), (Tabata et al. 1997). The paramagnetic fullerene can also be built into water-soluble dendrimers and the like. (Reviewed by Hirsch 1994).

Moreover, Boulas et al., J. Phys. Chem., 98, 1282-1287 (1993) disclose a method for increasing the water solubility of fullerene molecules and ions by forming inclusion complexes of fullerenes within cyclodextrin molecules.

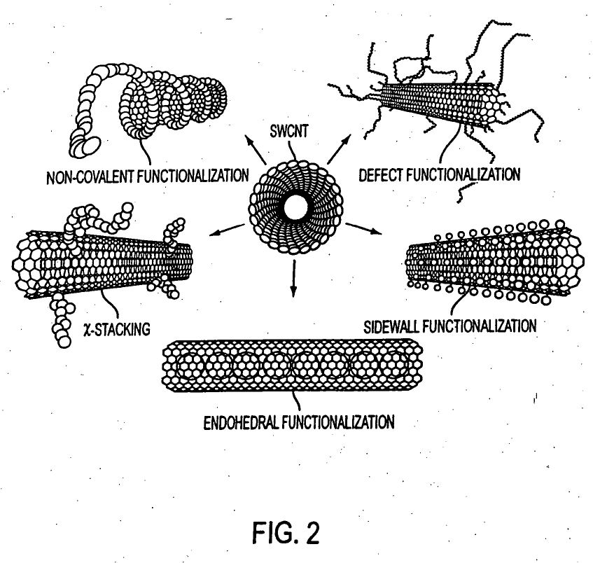

FIG. 2 represents various functionalization procedures for rendering the carbon-13 enriched CNT water soluble. Various embodiments of the CNT functionalizations include non-covalent, defect, sidewall, π-stacking and endohedral. It is also possible to produce adducts to improve water solubility by surface functionalization by derivatizing it with water soluble adducts such as hydroxyl and carboxyl groups as described above.

However, the present invention is not limited to the literature methods and could include various other groups on or in the present water-soluble fullerenes and CNTs without departing from the scope of the present invention.

According to another embodiment of the present invention the water soluble carbon-13 enriched fullerenes and CNTs are enhanced for imaging studies using the method of DNP. Direct ¹³C spectroscopy and imaging would be of little use because the carbon-13 signal even if it is isotopically enriched produces inherently weak signal. However, the sensitivity of the MRI signal is enhanced several fold (about 10³) by using the technique of dynamic nuclear polarization (DNP), also called “Overhauser effect.” This technique has been described in complete detail in the '814 patent of Ardenkjaer et al and the main embodiments of the technique are highlighted in this disclosure. In this method, the enhancement arises from the enhanced polarization of nuclear spins due to the transfer of the larger electron spin polarization through microwave radiation at or near the electron paramagentic resonance frequency. Thus, this invention preferably achieves an ex-vivo polarization by using a polarizing paramagnetic species such as MnCl₂ (Mn²⁺), FeCl₃ (Fe³⁺) or organic radicals or hyperpolarizable noble gases such as ³He and ¹²⁹Xe (OMRI agents) in the vicinity of the carbon-13 enriched fullerenes and CNTs.

The main advantage of using the molecules described herein is the number of “identical” carbons in a single molecule. To be an advantage means that it has to be enriched to greater than one ¹³C per molecule (e.g. 1/60˜1.7%), but in practice, there should be no disadvantage to using 100%. The present invention also includes molecules with 2 ¹³C per molecule (e.g. 2/60˜3.3%), 3 ¹³C per molecule e.g. 3/60˜5.0%), 4 ¹³C per molecule e.g. 4/60˜6.6%), 5 ¹³C per molecule (e.g. 5/60˜8.3%), 6 ¹³C per molecule (e.g. 6/60˜10.0%), 7 ¹³C per molecule (e.g. 7/60˜11.6%), 8 ¹³C per molecule (e.g. 8/60˜13.3%), 9 ¹³C per molecule (e.g. 9/60˜15%), and/or 10 ¹³C per molecule (e.g. 10/60˜16.6%). The present invention also includes molecules with from about 1-10 ¹³C per molecule, 10-20 ¹³C per molecule, 20-30 ¹³C per molecule, 30-40 ¹³C per molecule, 40-50 ¹³C per molecule, and/or 50-60 ¹³C per molecule, and/or increments therein.

The technique of ex-vivo DNP is particularly suited to carbon-13 enriched fullerenes because it has a long T₁ relaxation time (carbon-spin lattice relation time, which is held to range from ˜2-100 s depending upon the temperature and viscosity). Thus, owing to this high T₁ relaxation time, once the fullerene is polarized, it will remain so for a sufficiently long time to allow the imaging procedure to be carried out in a fairly comfortable time span.

Hyperpolarization may be carried out by three possible mechanisms: (1) the Overhauser effect, (2) the solid effect and (3) thermal mixing effect (see A. Abragam and M. Goldman, Nuclear Magnetism: Order and Disorder, Oxford University Press, 1982). By hyperpolarization, it is meant that the sample is polarized to a level over that found at room temperature and 1 T, preferably polarized to a polarization degree in excess of 0.1%, more preferably 1%, even more preferably 10%. The Overhauser effect is the preferred method of the present invention though other methods are also anticipated. It is envisaged that, in the method according to the invention, the level of polarization achieved should be sufficient to allow the hyperpolarized solution of the carbon-13 enriched fullerenes and CNTs to achieve a diagnostically effective contrast enhancement in the sample to which it is subsequently administered in whatever form. In general, it is desirable to achieve a level of polarization which is at least a factor of 2 or more above the field in which MRI is performed, preferably a factor of 10 or more, particularly preferably 100 or more and especially preferably 1000 or more, e.g. 50000.

In another embodiment of the method according to the present invention, hyperpolarization of the MR imaging nuclei is effected by a DNP free radical source. In this embodiment, step (i) of the method comprises: (a) bringing an DNP free radical source and the carbon-13 enriched fullerene and CNTs into contact in a uniform magnetic field (the primary magnetic field Bₒ); (b) exposing said DNP free radical source to a first radiation of a frequency selected to excite electron spin transitions in said DNP free radical source; and (c) dissolving in a physiologically tolerable solvent said carbon-13 enriched fullerenes and CNTs. It is preferred that the DNP free radical source and carbon-13 enriched fullerene and CNTs are present as a composition during polarization.

For the purposes of administration, the carbon-13 enriched fullerenes and CNTs should preferably be administered in the absence of the whole of, or substantially the whole of, the DNP free radical source. Preferably, at least 80% of the DNP free radical source is removed, at least 85% of the DNP free radical source is removed, particularly preferably 90% or more, especially preferably 95% or more, most especially 99% or more. In general, it is desirable to remove as much DNP free radical source as possible prior to administration to improve physiological tolerability and to increase T₁. Thus, preferred DNP free radical source for use in the first embodiment of the method according to the present invention are those which can be conveniently and rapidly separated from the polarized carbon-13 enriched fullerene and nanotubes MR imaging agent using known techniques.

However, where the DNP free radical source is non-toxic, the separation step may be omitted. A solid (e.g. frozen) composition comprising an DNP free radical source and carbon-13 enriched fullerene or CNT agent which has been subjected to polarization may be rapidly dissolved in saline (e.g. warm saline) and the mixture injected shortly thereafter.

Unless the hyperpolarized agent is stored (and/or transported) at low temperature and in an applied field as described above, since the method of the invention should be carried out within the time that the hyperpolarised solution of the carbon-13 enriched fullerene or nanotube agent remains significantly polarized, it is desirable for administration of the polarized carbon-13 enriched fullerene and nanotubes MRI agent to be effected rapidly and for the MR measurement to follow shortly thereafter.

It is envisaged that in one of the embodiments of the method according to the present invention, use may be made of any known DNP free radical source capable of polarizing a carbon-13 enriched fullerene or nanotube agent to an extent such that a diagnostically effective contrast enhancement, in the sample to which the carbon-13 enriched fullerene or nanotube agent is administered, is achieved.

In a preferred embodiment paramagnetic metal complexes are used. For example, these metal ions are chromium (III), manganese (II), manganese (III), iron (III), praseodymium (III), neodymium (III), samarium (III), ytterbium (III), gadolinium (III), terbium (III), dysprosium (III), holmium (III), or erbium (III).

Where the DNP free radical source is a paramagnetic free radical, the radical may be conveniently prepared in situ from a stable radical precursor by a conventional physical or chemical radical generation step shortly before polarization, or alternatively by the use of ionizing radiation. This is particularly important where the radical has a short half-life. In these cases, the radical will normally be non-reusable and may conveniently be discarded once the separation step of the method according to the invention has been completed.

Preferably, a chosen DNP free radical source will exhibit a long half-life (preferably at least one hour), long relaxation times (T₁ₑ and T₂ₑ), high relaxivity and a small number of ESR transition lines. Thus the paramagnetic oxygen-based, sulphur-based or carbon-based organic free radicals or magnetic particles referred to in WO-A-88/10419, WO-A-90/00904, WO-A-91/12024, WO-A-93/02711 or WO-A-96/39367 would also be suitable DNP free radical source in this invention.

In another embodiment, DNP free radical source suitable for use in this invention include hyperpolarizable gases. By hyperpolarisable gas, it is meant a gas with a nonzero spin angular momentum capable of undergoing an electron transition to an excited electron state and thereafter of decaying back to the ground state. Depending on the transition that is optically pumped and the helicity of the light a positive or negative spin hyperpolarisation may be achieved (up to 100%). Examples of gases suitable for use in the method of the invention include the noble gases He (e.g., ³He or ⁴He), Ne, Ar, Kr and Xe (e.g. ¹²⁹Xe), preferably He, Ne or Xe, especially preferably He, particularly ³He. Alkali metal vapors may also be used, e.g., Na, K, Rb, Cs vapors. Mixtures of the gases may also be used. In one embodiment of the method of the invention, the hyperolarizable gas may be used in liquid form.

In the separation step of the present invention of the method of the invention, it is desirable to remove substantially the whole of the DNP free radical source from the composition (or at least to reduce it to physiologically tolerable levels) as rapidly as possible. Many physical and chemical separation or extraction techniques are known in the art and may be employed to effect rapid and efficient separation of the DNP free radical source and carbon-13 enriched fullerene and nanotubes agent. Clearly, the more preferred separation techniques are those which can be effected rapidly and particularly those which allow separation in less than one second. In this respect, magnetic particles (e.g., superparamagnetic particles) may be advantageously used as the DNP free radical source as it will be possible to make use of the inherent magnetic properties of the particles to achieve rapid separation by known techniques. Similarly, where the DNP free radical source or the particle is bound to a solid bead, it may be conveniently separated from the liquid (i.e., if the solid bead is magnetic by an appropriately applied magnetic field).

For ease of separation of the DNP free radical source and the carbon-13 enriched fullerene and nanotubes agent, it is particularly preferred that the combination of the two be a heterogeneous system, e.g., a two phase liquid, a solid in liquid suspension or a relatively high surface area solid substrate within a liquid, e.g., a solid in the form of beads, fibers or sheets disposed within a liquid phase carbon-13 enriched fullerene and nanotubes agent. In all cases, the diffusion distance between the carbon-13 enriched fullerene or nanotube agent and DNP free radical source must be small enough to achieve an effective Overhauser enhancement. Certain DNP free radical source are inherently particulate in nature, e.g., the paramagnetic particles and superparamagnetic agents referred to above. Others may be immobilized on, absorbed in or coupled to a solid substrate or support (e.g., an organic polymer or inorganic matrix such as a zeolite or a silicon material) by conventional means. Strong covalent binding between DNP free radical source and solid substrate or support will, in general, limit the effectiveness of the agent in achieving the desired Overhauser effect and so it is preferred that the binding, if any, between the DNP free radical source and the solid support or substrate is weak so that the DNP free radical source is still capable of free rotation. The DNP free radical source may be bound to a water insoluble substrate/support prior to the polarization or the DNP free radical source may be attached/bound to the substrate/support after polarization. The DNP free radical source may then be separated from the carbon-13 enriched fullerene and nanotubes agent, e.g., by filtration before administration. The DNP free radical source may also be bound to a water soluble macromolecule and the DNP free radical source-macromolecule may be separated from the carbon-13 enriched fullerene or CNT agent before administration.

Where the combination of an DNP free radical source and carbon-13 enriched fullerene or CNT agent is a heterogeneous system, it will be possible to use the different physical properties of the phases to carry out separation by conventional techniques. For example, where one phase is aqueous and the other non-aqueous (solid or liquid) it may be possible to simply decant one phase from the other. Alternatively, where the DNP free radical source is a solid or solid substrate (e.g., a bead) suspended in a liquid carbon-13 enriched fullerene or nanotube agent the solid may be separated from the liquid by conventional means, e.g., filtration, gravimetric, chromatographic or centrifugal means. It is also envisaged that the DNP free radical source may comprise lipophilic moieties and so be separated from the carbon-13 enriched fullerene and CNT by passage over or through a fixed lipophilic medium or the DNP free radical source may be chemically bound to a lipophilic solid bead. The carbon-13 enriched fullerene and CNT agent may also be in a solid (e.g., frozen) state during polarization and in close contact with a solid DNP free radical source. After polarization it may be dissolved in heated water or saline or melted and removed or separated from the DNP free radical source where the latter may be toxic and cannot be administered.

The preferred administration route for the polarized carbon-13 enriched MRI agent is parenteral, e.g., by bolus injection, by intravenous, intraarterial or peroral injection. The injection time should be equivalent to 5 T₁ or less, preferably 3 T₁ or less, particularly preferably T₁ or less, especially 0.1 T₁ or less. The lungs may be imaged by spray, e.g., by aerosol spray. Parenteral compositions may be injected directly or mixed with a large volume parenteral composition for systemic administration. Formulations for enteral administration may vary widely, as is well-known in the art. In general, such formulations include a diagnostically effective amount of the carbon cluster derivatives. Such enteral compositions may optionally include buffers, surfactants, thixotropic agents, and the like. Compositions for oral administration may also contain flavoring agents and other ingredients for enhancing their organoleptic qualities.

The diagnostic compositions are administered in doses effective to achieve the desired enhancement of the NMR image. Such doses may vary widely, depending upon the percentage of carbon-13 enrichment, the organs or tissues which are the subject of the imaging procedure, the NMR imaging equipment being used, etc. The diagnostic compositions of this invention are used in a conventional manner in magnetic resonance procedures. Compositions may be administered in a sufficient amount to provide adequate visualization, to a warm-blooded mammal either systemically or locally to an organ or tissues to be imaged, and the mammal then subjected to the MRI procedure. The compositions enhance the magnetic resonance images obtained by these procedures.

Another embodiment encompasses any method that would polarize the free radical agents described herein over thermal equilibrium (e.g., storing the compound at low temperature and high field).

In another embodiment, the general protocol comprises polarizing and solublizing the molecule in a magnet, where the radical is filtered out, and a quality control (temperature, pH, polarization) is made quickly followed by intravascular injection.

### EXAMPLES

To a sample of solubilized carbon 13 enriched fullerene or CNT is added a water soluble free radical source (0.1%), cooled to 4.2 K and placed in a 2.5 T magnetic field.

The sample is polarized by microwaves (70 GHz) for at least 1 hour at a field of 2.5 T at a temperature of 4.2 K. The progress of the polarization process is followed by in situ NMR (fast adiabatic passage). When a suitable level of polarization has been reached, the ampule is rapidly removed from the polarizer and, while handled in a magnetic field of no less than 50 mT, cracked open and the contents are quickly discharged and dissolved in warm (160° C.) water.

Experiment 1: This solution is quickly transferred to a spectrometer and carbon-13 spectrum with enhanced intensity is recorded.

Experiment 2: The sample solution is inserted into an MRI machine with carbon-13 measurement capability and a picture with enhanced intensity and contrast is obtained by a single shot technique.

Experiment 3: The solution is quickly injected into a mammal, e.g. a rat, and a carbon-13 MRI picture with enhanced intensity and contrast is obtained, also in this case, by utilization of a single shot technique.

The embodiments disclosed herein have novel medical utility and applications as enhanced molecular imaging beacons in MRI. More specifically, these class of compounds have exceptional advantages for MRI studies in targeting and as blood pool imaging agents as described above.

The particular embodiments described herein are illustrative and representative and are not meant to be limiting. From consideration and applications of the invention disclosed herein, it will be apparent to those skilled in the art that various changes and modifications could be made and similar advantages over the existing art could be obtained by other embodiments. Any such modifications of the present invention which comes within the spirit and scope of the invention is considered to be part of this invention.

What is claimed is:

1. A contrast agent for enhancing contrast in in vivo magnetic resonance imaging measurements, comprising an air stable, water soluble carbon-13 enriched fullerene or carbon nanotube molecule.

2. The agent according to claim 1 wherein said fullerene has a solubility in water of at least 1 mM.

3. The agent according to claim 1 wherein the fullerene has carbon-13 isotope abundance in the range of 10-100%.

4. A method of water solubilizing a carbon-13 enriched fullerene or carbon nanotube contrast agent comprising derivatizing the fullerene shell with water soluble functional groups.

5. The method according to claim 4 wherein the functional groups comprise carboxyl groups, hydroxyl groups, or amino acid groups.

6. A dynamic nuclear polarization (DNP) method of magnetic resonance imaging (MRI) measurement of a sample of body organs and tissues, comprising:
(i) producing a hyperpolarized solution of carbon-13 enriched fullerenes or carbon nanotubes wherein the hyperpolarization of the sample is effected by means of a polarizing agent;
(ii) optionally separating the whole, substantially the whole, or a portion of said polarizing agent from the carbon-13 enriched fullerenes or carbon nanotubes;
(iii) administering to said sample a diagnostically effective amount of the hyperpolarized solution;
(iv) exposing said sample to radiation of a frequency selected to excite nuclear spin transitions in magnetic resonance imaging carbon-13 nuclei of the enriched fullerenes or carbon nanotubes; and
(v) detecting magnetic resonance signals from said sample.

7. The method according claim 6, further comprising generating an image, dynamic flow data, diffusion data, or perfusion data from the detected signals.

8. The method according to claim 6 wherein step (i) comprises hyperpolarizing the solid carbon-13 enriched fullerene or CNT by irradiating a polarizing agent to cause dynamic nuclear polarization.

9. The method according to claim 6 wherein the polarizing agent is a paramagnetic metal species or a paramagnetic free radical or a hyperpolarized gas.

10. The method according to claim 9 wherein the paramagnetic metal species is chromium (III), manganese (II), manganese (III), iron (III), praseodymium (III), neodymium (III), samarium (III), ytterbium (III), gadolinium (III), terbium (III), dysprosium (III), holmium (III, or erbium (III).

11. The method according to claim 9 wherein the hyperpolarized gas is ¹²⁹Xe, ³He or ⁴He.

12. A method for providing diagnostic treatment to a patient comprising administering to said patient a diagnostically effective amount of a composition comprising carbon-13 enriched water soluble fullerene or carbon nanotube MRI contrast agents.

13. A method of using carbon-13 enriched fullerenes or carbon nanotubes for an in vivo MRI procedure in blood pool imaging or targeting studies.

14. A pre-polarized or hyperpolarized composition of carbon-13 enriched fullerenes or carbon nanotubes.

15. The composition according to claim 14, wherein the hyperpolarization is effected by an ex-vivo dynamic nuclear polarization (DNP) technique using an OMRI reagent.
